(19) United States
(12) Patent Application Publication
(10) Pub. No.: US 2012/0035652 A1
(43) Pub. Date: Feb. 9, 2012

(54) Sciatic Relief Device

(76) Inventor: William A. McGrimley, Seneca Falls, NY (US)

(21) Appl. No.: 12/851,116

(22) Filed: Aug. 5, 2010

(51) Int. Cl. A61B 17/00

(52) U.S. Cl. 606/201

(57) ABSTRACT

A sciatic relief device comprising a base, at least one reversibly inflatable bladder enclosed in a sleeve, and an inflation device attached to the bladder. A method of use for the sciatic relief device, wherein a user positions his thigh on top of the inflated bladder for at least a short period of time to alleviate the pain and other symptoms associated with sciatica.

### BACKGROUND OF THE INVENTION

1. Field of the Invention

The present invention relates to a device for relieving pain, and, more specifically, to a device for relieving pain caused by the sciatic nerve.

2. Description of the Related Art

Sciatica is the name given to the symptoms caused by compression or irritation of the sciatic nerve, including pain, numbness, and weakness. The sciatic nerve, which is derived from spinal nerves L4 through S3, is the longest nerve in the human body, extending from the lower back through the buttock down the leg.

Sciatica is commonly caused by compression or irritation of the sciatic nerve, typically due to a bulging or herniated disc in the spine. Pain associated with sciatica is typically felt in the lower back, the buttocks, or the legs and feet. Although the exact number of people suffering from sciatica is currently unknown, it is estimated that millions of people around the world exhibit one or more symptoms associated with the condition.

Treatment of sciatic includes such methods as medication with a pain-reliever or anti-inflammatory, physical therapy, acupuncture, and massage therapy. Unfortunately, many methods of treatment are ineffective, laden with side effects, or prohibitively expensive. There remains, therefore, a continued need for an effective, inexpensive treatment for the symptoms associated with sciatica.

### BRIEF SUMMARY OF THE INVENTION

It is therefore a principal object and advantage of the present invention to provide a device for the treatment of sciatica.

It is another object and advantage of the present invention to provide a simple and effective device for the treatment of sciatica symptoms.

It is yet another object and advantage of the present invention to provide an affordable device for the treatment of sciatica.

Other objects and advantages of the present invention will in part be obvious, and in part appear hereinafter.

In accordance with the foregoing objects and advantages, the present invention provides a sciatic relief device comprising the following: (i) a base; (ii) a reversibly inflatable bladder that defines a groove along a longitudinal axis of the device; (iii) a sleeve at least partially surrounding the bladder at least partially attached to the base; (iv) inflation means; and (v) a tube connected the bladder at one end and connected to the inflation means at the other end.

A second aspect of the present invention provides a sciatic relief device with a bladder that has first and second chambers that define a groove running along a longitudinal axis of the device.

A third aspect of the present invention provides a sciatic relief device that includes deflation means such as a pressure-release valve or a manual-release valve.

A fourth aspect of the present invention provides a sciatic relief device that includes heating means, such as a heating coil, and/or cooling means, such as a coolant. The heating and cooling means can remote from the device or can be an integral part of the device including part of the bladder(s), the base, or the sleeve, among other locations.

A fifth aspect of the present invention provides a method of treating a user's sciatica symptoms, the method comprising the steps of: (i) positioning a region of the user's body in the groove of the sciatic relief pad; and (ii) maintaining the body part in the groove for a period of time. The most common regions include the gluteal region and the right or left thigh region. The method can optionally include the step of adjusting the inflation of the bladder, cooling the user's body part, and/or heating the user's body part.

### BRIEF DESCRIPTION OF THE SEVERAL VIEWS OF THE DRAWING(S)

The present invention will be more fully understood and appreciated by reading the following Detailed. Description in conjunction with the accompanying drawings, in which:

FIG. 1 is a schematic drawing depicting a top view of the device according to one embodiment of the present invention;

FIG. 2 is a schematic drawing depicting a side section view of the device according to one embodiment of the present invention;

FIG. 3 is a schematic drawing depicting an enlargement of the circled section in FIG. 2;

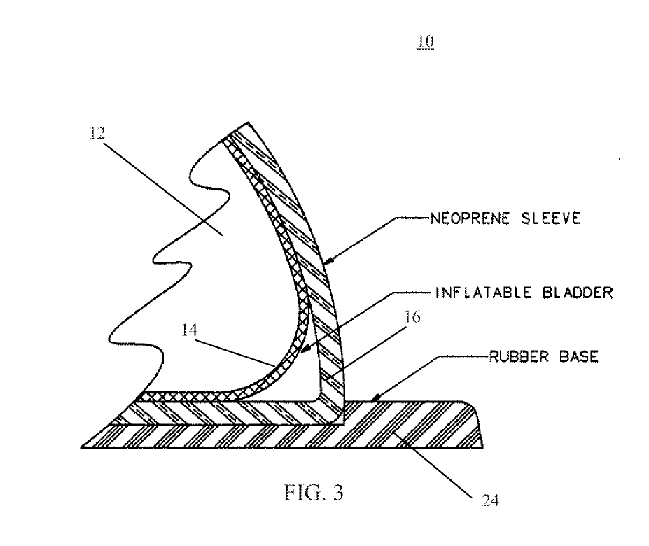

FIG. 4 is a schematic drawing depicting an end view of the device according to one embodiment of the present invention;

FIG. 5 is a schematic drawing depicting an inflation bulb according to one embodiment of the present invention;

FIG. 6 is a schematic drawing depicting a top view of the device and a rubber base of the device according to one embodiment of the present invention;

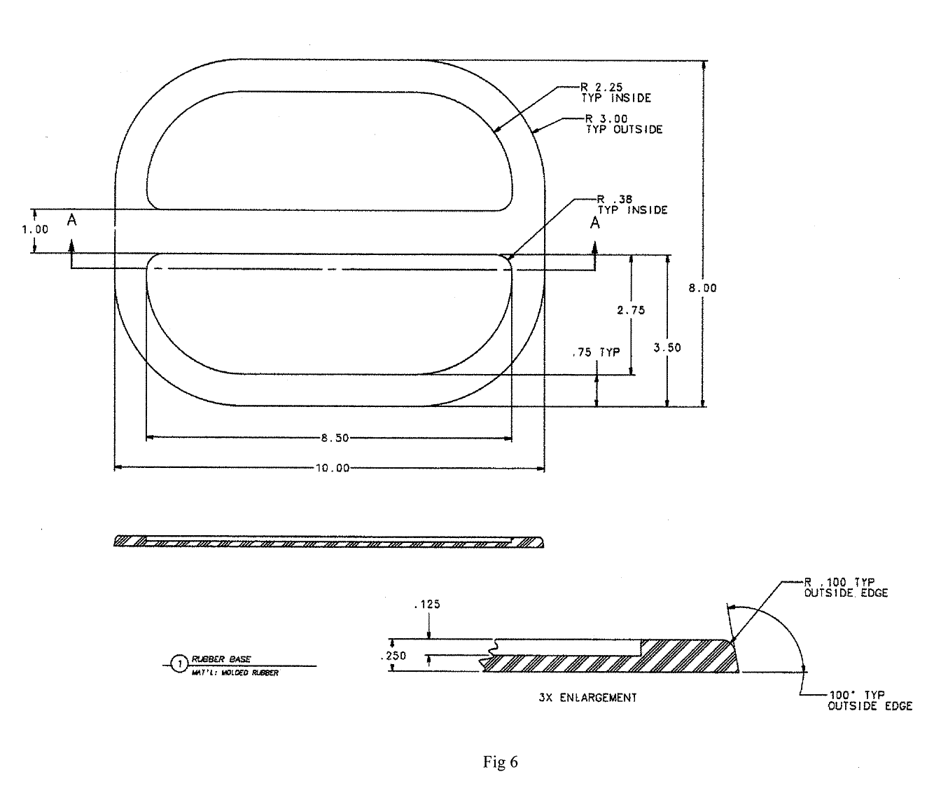

FIG. 7 is a schematic drawing depicting an inflatable bladder of the device according to one embodiment of the present invention; and

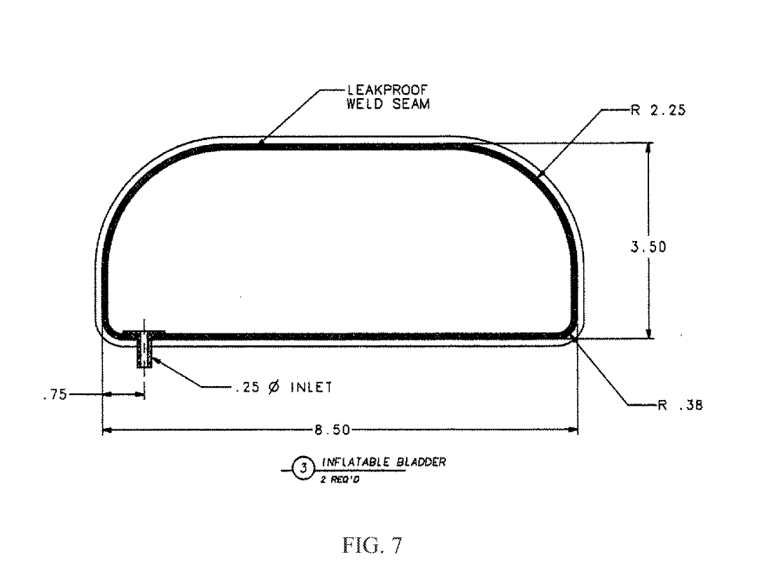

FIG. 8 is a schematic drawing depicting a sleeve surrounding an inflatable bladder of the device according to one embodiment of the present invention.

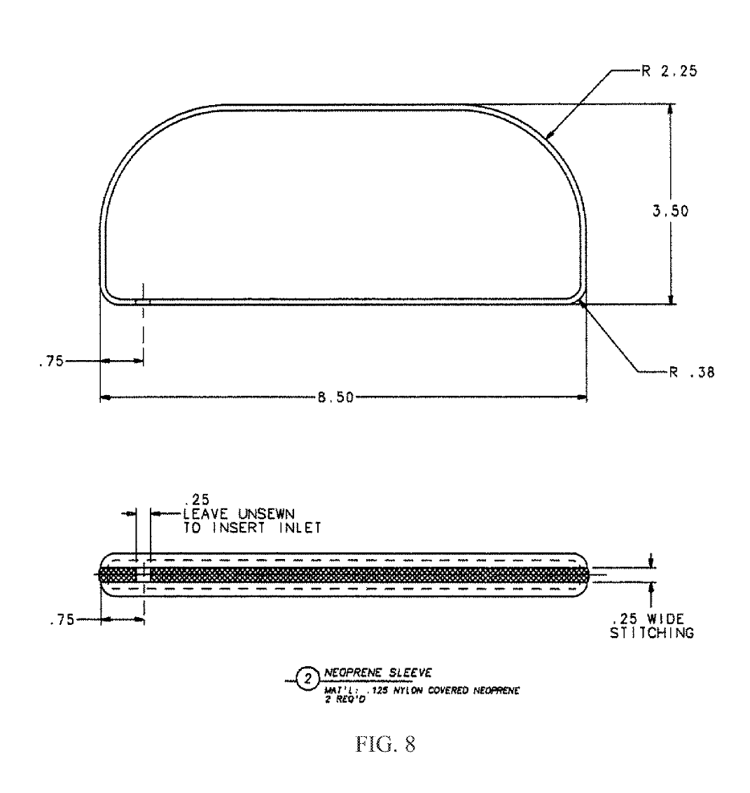

### DETAILED DESCRIPTION OF THE INVENTION

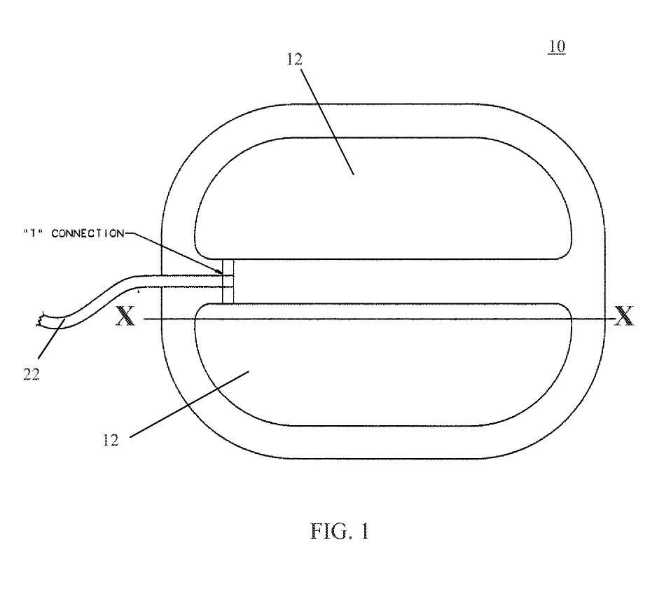

Referring now to the drawings, wherein like reference numerals refer to like parts throughout, there is seen in FIG. 1 a schematic drawing depicting a top view of the sciatic relief device 10 according to one embodiment of the present invention. Sciatic relief device 10 comprises two inflatable bladders 12. In a preferred embodiment inflatable bladders 12 are filled with a gas such as air or an inert gas, but in other embodiments the bladders can be filled with a liquid, including but not limited to water, or a gel. The walls 14 of the inflatable bladders comprise a resilient but flexible material that allows the bladder to increase in size when filled with a gas, liquid, or gel, but does not burst when exposed to the operating weight. The flexible material can be any material with this desired elastomeric property, including an elastic hydrocarbon polymer such as a rubber, of which there are many natural and synthetic varieties.

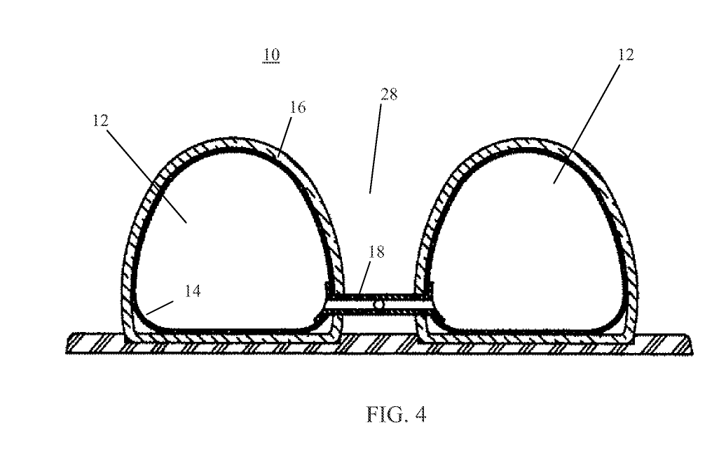

The inflatable bladders can comprise a variety of shapes, sizes, and configurations. In one embodiment, the bladders comprise a single inflatable chamber such that the bladder has one access point for the entry of air or liquid for inflation and deflation; air or liquid entering through the access point will have the choice of entering either side of the single inflatable bladder, as suggested in FIG. 4. In another configuration, the bladders comprise two or more separate bladders, and each can optionally contain a separate access point for inflation.

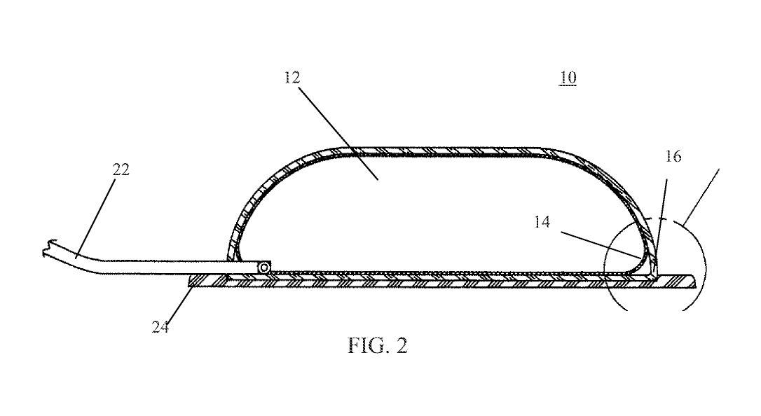

In a preferred embodiment, inflatable bladders 12 are further surrounded by a sleeve 16, as shown in FIG. 2. The sleeve helps the bladder retain its shape and keeps the bladder positioned properly within sciatic relief device 10 even when the bladder is repeatedly deflated and inflated. Sleeve 16 can comprise a unified piece of material encapsulating one or more bladders on both sides of device 10. Sleeve 16 can also contain more than one inflatable bladder 12, depending upon the particular configuration of the device. The sleeve is preferably permanently attached to a base 24, although sleeve 16 could be attached to base 24 by reversible means such as a hook-and-latch system, tape, or other means known in the art. In a preferred embodiment, base 24 comprises a solid material such as plastic, wood, leather, neoprene, or rubber, among others, that allows device 10 to sit flat on a surface.

In yet another embodiment, sleeve 16 can be lined—either partially or completely—with a soft and/or absorbent material (not shown). The lining can provide comfort, padding, or absorbency to improve the user experience during use.

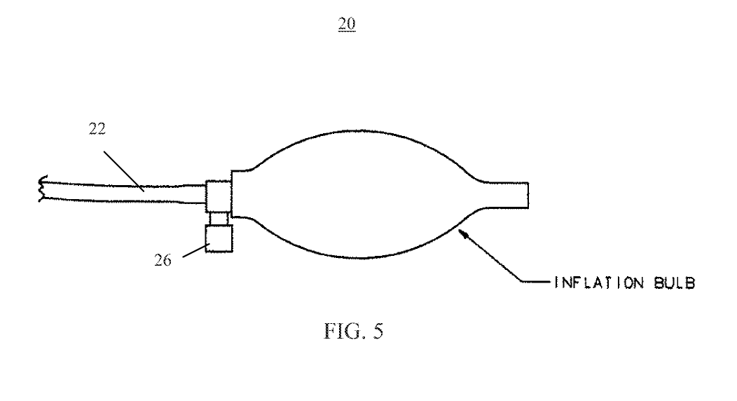

Sciatic relief device 10 further comprises inflation means 20 connected to the device by a hollow tube 22. Hollow tube 22 connects to the inflation means at one end and connects to the inflatable bladders of device 10 at the other end. The inflation means forces air or liquid through hollow tube 22 into the inflatable bladder. In a preferred embodiment, inflation means 20 is an inflation bulb, as shown in FIG. 5, although the inflation means can be mechanical means such as a electric-powered pump. Inflation bulb 20 is hand-operated; squeezing the bulb forces air into tube 22 and therefore into the inflatable bladders. The inflation bulb further comprises deflation means 26, which in FIG. 5 is a pressure-release button that allows air to exit from device 10. The deflation means could also be a hand-turned valve that allows air to exit from the device when it is manually opened. In another embodiment, hollow tube 22 is mounted on device 10 at a location other than the location depicted in FIG. 1, including either end of device 10 or along the top, bottom, or sides of device 10 depending upon the particular design and/or needs of the user.

In a preferred embodiment, sciatic relief device 10 is used to alleviate pain and/or other symptoms associated with sciatica. To relieve symptoms, the user first places base 24 of device 10 on a substantially flat surface such as a floor, chair, couch, or other place suitable for sitting or resting. The user then inflates the inflatable bladders or ensures that the bladders are still inflated from a prior use. The user can optionally inflate the bladders to a level that is maximally comfortable or has previously been shown to best alleviate pain and/or other symptoms. Additionally, the user can adjust the inflation of the bladders during use. Once the bladders are inflated, the user places his or her thigh or gluteal region, for example, on top of the device, with the user's femur oriented along the X-X axis, shown in FIG. 1. Preferably, the user's body part fits comfortably into the valley-like region or channel 28 formed by the inflated bladders, although the body part will be slightly elevated through interaction with the bladder on each side rather than fit firmly against base 24 between the bladders. Through the comfortable elevation of the body part resting on the bladders, device 10 alleviates the pain and/or other symptoms associated with sciatica. It should be noted, however, that the user can adjust one or more of the inflatable bladders 12 as well as the orientation of device 10, among other adjustments, to achieve maximum relief of the sciatica symptoms.

In a preferred embodiment, device 10 fits all ages and sizes of people through the adjustability of the inflatable bladders. In another embodiment the specifications of device 10, including the spacing of the bladders and other variables, are adjusted based on the planned user. For example, smaller bladders are used for devices marketed to children and small adults. Larger, more robust bladders can be used for devices marketed to adults. Preferably, the bladders are spaced apart to define a channel of about one inch for an average size person. Although the width of the channel may vary, the existence of the channel is critical to providing relief from sciatica.

Sciatic relief device 10 can include other options that enhance the relief of sciatica symptoms. For example, the device can include heating coils inside the bladder, the walls of the bladder, the base, or the lining, among other locations, which heat the device during use. The heat will radiate upwardly from the device into the thigh of the user, further alleviating symptoms. In yet another embodiment, the device includes cooling means, such as a coolant, to cool the user's body part. The coolant can be an ice pack, a refrigerated coil, or any other known means of cooling.

Although the present invention has been described in connection with a preferred embodiment, it should be understood that modifications, alterations, and additions can be made to the invention without departing from the scope of the invention as defined by the claims.

What is claimed is:

1. A sciatic relief device, comprising:
a bladder comprising first and second inflatable chambers separated apart from each other by a non-inflatable channel; and
a sleeve at least partially surrounding said inflatable bladder.

2. The sciatic relief device of claim 1, wherein said first and second inflatable chambers and said channel are dimensioned to accommodate a human leg.

3. The sciatic relief device of claim 1, further comprising means for inflating and deflating said bladder.

4. The sciatic relief device of claim 1, wherein said means for inflating and deflating said bladder comprises a valve and a pump.

5. The sciatic relief device of claim 4, wherein said valve comprises a pressure-release valve.

6. The sciatic relief device of claim 4, wherein said valve comprises a manually-released valve.

7. The sciatic relief device of claim 1, wherein said bladder is filled with a fluid selected from the group comprising a liquid, a gel, and a gas.

8. The sciatic relief device of claim 1, wherein said sleeve is padded.

9. The sciatic relief device of claim 1, further comprising a heat source.

10. The sciatic relief device of claim 9, wherein said heating source comprises a heating coil positioned in proximity to said bladder.

11. The sciatic relief device of claim 1, further comprising a cooling source.

12. The sciatic relief device of claim 11, wherein said cooling source comprises coolant in fluid communication with said bladder.

13. A method of treating sciatica symptoms in a person, comprising the steps of:
providing a bladder comprising first and second inflatable chambers separated apart from each other by a non-inflatable channel;
inflating said bladder; and
positioning a region of said person at least partially in the channel so that said first and second inflatable chambers provide support on either side of said region.

14. The method of claim 13, wherein said region is a thigh.

15. The method of claim 13, wherein said thigh extending longitudinally along the same axis as said channel.

16. The method of claim 13, wherein said region is a gluteal region.

17. The method of claim 13, further comprising the step of adjusting the inflation of said bladder.

18. The method of claim 13, further comprising the step of cooling said region by cooling said bladder.

19. The method of claim 13, further comprising the step of heating said region by heating said bladder.
